(19) United States
(12) Patent Application Publication
(10) Pub. No.: US 2006/0049050 A1
(43) Pub. Date: Mar. 9, 2006

(54) Separation of molecules

(76) Inventors: Michel Daniel Faupel, Echentzwiller (FR); Patrick Andre Schindler, Muespach (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/516,029

(22) PCT Filed: May 30, 2003
(86) PCT No.: PCT/EP03/05704

§ 371 Date: May 20, 2005

(30) Foreign Application Priority Data
Jun. 1, 2002 (GB) 0212853.6

(51) Int. Cl. C07K 1/26; G01N 27/447

(52) U.S. Cl. 204/450; 204/600

(57) ABSTRACT

The present invention relates to a method and an apparatus for separation of molecules, particularly biomolecules, in solution. Certain aspects of the invention further relate to a system for automated separation of molecules in solution. Further aspects of the invention relate to a computer program for separation of molecules in solution.

### SEPARATION OF MOLECULES

The present invention relates to a method and an apparatus for separation of molecules, particularly biomolecules, in solution. Certain aspects of the invention further relate to a system for automated separation of molecules in solution. Further aspects of the invention relate to a computer program for separation of molecules in solution.

For many purposes, it is desirable to be able to separate particular molecules from a mixture of molecules. For example, for purification of proteins or other biomolecules from cell extracts; for purification of synthesised chemicals from contaminants; or for separation of mixtures of chemicals, separation of molecules is desirable. Further, separation of molecules may also be desirable for analysis or identification of components of a mixture of molecules. In particular, the growing science of proteomics requires the separation and identification of many of the molecules within a cell's proteome (that is, the complete complement of proteins produced by a particular cell).

Traditionally such separation or purification has been carried out by means of electrophoresis; that is, the separation of molecules according to the charge carried by the molecule. The charge carried by the molecule may be varied by varying the conditions under which electrophoresis is performed; thus, the separation may be ‘fine-tuned’ depending on the types of molecule to be separated. Many types of electrophoresis are generally carried out within a solid medium, such as agarose or polyacrylamide gel. While this is relatively simple and effective, it does require additional steps subsequent to separation should the separated molecules need to be recovered from the solid medium. These additional steps may be time-consuming, require the use of additional reagents, and any additional manipulation steps may increase the risk of damaging the molecule being recovered.

Accordingly, it is desirable for certain types of electrophoresis to be conducted in liquid supports. In particular, the technique of isoelectric focusing (IEF) may be conducted in a liquid medium; for example, as described in U.S. Pat. No. 4,971,670.

During IEF in a liquid medium, a pH gradient is established along the medium by the use of a series of graduated membranes of differing pH, which partition the medium into a series of compartments creating the pH gradient. When an electric field is applied to the liquid medium, charged molecules in the medium migrate through the medium and pass through the membranes to the isoelectric point of the molecules. In this way, a mixture of molecules may be separated according to their charge under the conditions used.

However, a number of different molecules may share the same isoelectric point, while differing in other characteristics. Thus, a single fraction separated by conventional IEF techniques may nonetheless still contain a mixture of molecules. Should a researcher wish to isolate a single molecular species from this mixture, it is necessary to perform additional processing and purification steps to identify and isolate the desired species from the mixture (for example, affinity binding, precipitation, or the like), thereby losing one of the chief advantages of IEF in a liquid medium as compared with IEF in a solid medium. Alternatively, of course, it is possible to perform two-dimensional (2-D) electrophoresis on a solid medium in order to isolate molecules of interest; again, however, subsequent processing and purification is necessary to recover an isolated molecule from the solid medium, so increasing the time and resources necessary to isolate the desired molecular species.

It is among the objects of embodiments of the present invention to obviate or alleviate these and other disadvantages of known IEF techniques. In particular, it is an object of certain embodiments of the invention to provide a method or an apparatus whereby multi-dimensional separation of molecules may be performed in a liquid medium.

According to a first aspect of the present invention, there is provided a method for separating molecules in a liquid medium, the method comprising the steps of:

locating a liquid medium containing molecules to be separated in a series of first fluid compartments, the first compartments being separated along a first axis by pH membranes to form a pH gradient along the series of fluid compartments, and at least one of said first compartments being adjacent a second compartment, said first and second compartment forming a second axis substantially perpendicular to the first axis;

applying a first electric field to the liquid medium along a first axis of the fluid compartments, thereby causing charged molecules in the liquid medium to migrate to their isoelectric point along the first axis; and

applying a second electric field to the liquid medium along said second axis, thereby causing charged molecules in the liquid medium to separate along said second axis according to a second characteristic of the molecules.

The method of the present invention thus allows molecules to be separated over two dimensions while remaining in a liquid medium. This greatly simplifies subsequent processing and recovery of the separated molecules, as well as simplifying the second separation, which may be carried out directly on the fluid as separated in the first dimension, rather than via an intermediate solid medium step.

An advantage of using a second dimension is that when the first dimension is carried out over multiple loading wells in parallel, the second dimension enables a concentrating effect without additional sample handling. This is of particular importance when detecting low abundance proteins or other molecules. Further, the second dimension may be used as a concentrating device; for example, the first dimension is carried out in parallel with multiple samples to obtain a particular pH range. The content of multiple identical wells may then be concentrated into a smaller volume by focusing in the second dimension between the same pH membranes as in the first dimension.

The second electric field may be applied sequentially or simultaneously with the first electric field. It is preferred that the second field is applied sequentially, since this allows the first separation to be completed before the second begins, thereby increasing the resolving power of the method.

The compartments along the second axis are preferably separated by membranes. The membranes may be pH membranes, to provide a second pH gradient; for example, this may be used to provide a finer resolution of separation within a particular pI range. Alternatively, the membranes along the second axis may be affinity membranes, antibody membranes, or the like, for binding particular components of the fluid. For example, the membranes may preferentially bind proteins or nucleic acids from the fluid. The method may further comprise the step of agitating or otherwise mixing the fluid, to encourage binding of fluid components to the membranes. Where the compartments are not separated by membranes, the electrical field will still separate molecules in accordance with their migration toward the cathode or anode.

The second axis may be formed by one or more second compartments adjacent the first compartment. In general, more compartments in the second axis allows greater resolution of molecules from the fluid.

One or more first compartments may be located adjacent second compartments to provide one or more second axes along which separations may take place. A plurality of second axes may be of use where multiple molecular species are to be separated; or where the user does not know in advance to which compartment a molecule of interest will separate along the first axis.

The second compartments along the second axis may initially be separated from the first compartments of the first axis by means of impermeable barriers. The method may then comprise the step of removing the impermeable barriers prior to applying the second electric field. The method may still further comprise the step of replacing the impermeable barriers with membranes. The method may yet further comprise the step of separating the first compartments along the first axis from the second compartments of the second axis by means of impermeable barriers.

The method may further comprise the step of applying an electric field to the liquid medium across a third axis substantially perpendicular to the second axis. This allows further separations to be conducted if desired. The third axis may be the same axis as the first axis; or the third axis may be parallel to the first axis; or the third axis may be perpendicular to the first axis. The particular configuration will depend on the nature of the separation being performed, as well as the preferences of the user.

The method may yet further comprise the step of recovering one or more separated fractions from one or more compartments. The recovery step may be automated or manual. The step may further comprise analysing or otherwise testing the recovered fraction to determine some characteristic thereof. Since the present invention allows the second separation to be performed in solution, the recovery step may be relatively straightforward, and may even comprise simply taking a fluid sample from the compartment.

According to a second aspect of the present invention, there is provided an apparatus for separating molecules in a liquid medium, the apparatus comprising a substrate defining a plurality of first fluid compartments arranged along a first axis, the first compartments being separated by pH membranes to form a pH gradient; at least one of the first compartments having a second compartment disposed adjacent thereto, said first and second compartments defining a second axis substantially perpendicular to the first axis; and at least two electrode pairs disposed across the first and second axes.

The compartments of the second axis may also be separated by membranes; these may be pH membranes or affinity membranes or the like.

The second compartments of the second axis may be separated from the first compartments of the first axis by removable impermeable barriers.

The second axis may comprise one or more second compartments disposed adjacent the or each of the first compartments of the first axis.

A plurality of first compartments of the first axis may have second compartments disposed adjacent thereto, to provide a plurality of second axes. A corresponding plurality of electrode pairs will also be provided.

The apparatus may include additional compartments disposed parallel to the first axis, to provide additional separation axes. These additional axes may be in fluid communication with the compartments of the first or second axes, or may be isolated therefrom, and simply provided on the same substrate. This allows multiple separations to be performed on the same substrate without interfering with one another.

The apparatus may further include a cover for location on the substrate. The cover may include means for engaging with the electrode pairs to provide electrical connections thereto. Alternatively, the electrode pairs may be provided on the cover, such that they extend into respective compartments provided on the substrate.

The apparatus preferably further includes fluid inlet and outlet channels or conduits. These may be defined by the substrate or cover alone or in co-operation.

The apparatus may yet further include means for supplying electric current to the electrode pairs, to apply an electric field to the apparatus. The current-supplying means may be selectively controllable, to supply current selectively, to allow for fine control of separation of molecules. The controllable current-supplying means may also be programmable, to provide current in a predetermined sequence or pattern.

According to a further aspect of the present invention, there is provided an apparatus for separation of molecules in a liquid medium, the apparatus comprising a substrate defining a plurality of first fluid compartments arranged along a first axis, at least one of the first compartments having a second compartment disposed adjacent thereto, said first and second compartment defining a second axis substantially perpendicular to the first axis; the first compartments along the first axis having means therebetween for receiving pH membranes to separate the first compartments; and the apparatus further comprising means for receiving at least two electrode pairs disposed along the first and second axes respectively.

The apparatus may further include means for receiving membranes located between the compartments of the second axis.

The means for receiving membranes may include means for receiving a removable cartridge holding a membrane. The apparatus may further include one or more removable cartridges holding a membrane, located within the means for receiving membranes.

The apparatus may yet further include one or more fluid impermeable barriers of suitable dimensions to be received within the means for receiving membranes.

The apparatus may further include a cover for location on the substrate.

An apparatus according to the present invention allows membranes to be removed and replaced relatively quickly and easily; in preferred embodiments of the invention, the cartridge form of the membranes is particularly convenient. This means that the apparatus may be rapidly reconfigured for different uses; for example, to alter the range of pH values across the pH gradient of the first axis.

The apparatus may further include one or more electrode pairs received within at least one of the electrode-receiving means. A single electrode pair may be provided, which may be reconfigured to allow an electric field to be applied along the second axis; although it is preferred that at least two electrode pairs are provided.

According to a further aspect of the present invention, there is provided a cartridge for use with an apparatus for separating molecules in a liquid medium, the cartridge comprising a frame defining an aperture, said aperture having a membrane therein, the frame further comprising a handling portion to permit handling of the cartridge.

Preferably the frame comprises two co-operating sections, which in use hold the membrane therebetween.

The frame may further define a second aperture having a second membrane therein; this allows rapid reuse of the cartridge without replacing a used membrane, or where the two membranes have different characteristics, rapid reconfiguration of the separation apparatus.

Preferably the frame is made of plastics material.

Preferably the membrane comprises polyacrylamide. Such membranes are known for use in IEF, and may have their pH fixed at a desired value, in accordance with techniques well known in the state of the art. Alternatively, the membrane may be any other suitable material, depending on the desired properties of the membrane; for example, nylon, cellulose, nitro-cellulose, or the like may be used. The membrane may further include additional chemical moieties bound onto the membrane surface; these may be selected to promote particular characteristics of the membrane or to enhance binding of desired molecules to the membrane.

The cartridge may further include polymeric or elastomeric seals for sealing the edges of the membrane from fluid penetration.

According to a still further aspect of the present invention, there is provided an automated system for separation of molecules from a liquid medium, the system comprising:

a substrate defining a plurality of first fluid compartments arranged along a first axis, the first compartments being separated by pH membranes to form a pH gradient; at least one of the first compartments having a second compartment disposed adjacent thereto, the first and second compartments defining a second axis substantially perpendicular to the first axis; and at least two electrode pairs disposed across the first and second axes;

control means for applying current selectively to the electrode pairs in a predetermined sequence; and

means for introducing and removing liquid containing molecules to be separated to and from the chambers.

The control means for selectively applying current may be an electricity supply including one or more electrical switches. The control means may further include a computer processor executing a suitable computer program; for example, particular desired durations and orientation of current to be applied may be programmed to be applied. The duration and orientation of current may be varied depending on the desired separation to be carried out. The computer processor may be specifically designed for executing the computer program, or a general purpose computer may be used.

The means for introducing and removing liquid may comprise fluid conduits or channels or the like, together with an appropriate fluid pump. The pump may also be controlled by a computer processor executing a suitable computer program, such that liquid is introduced and removed at appropriate times, depending on the separation being conducted.

The membranes of the system may be in the form of removable cartridges, allowing the membranes to be replaced or removed as necessary. Where this is the case, the system may further include automated means for removing or inserting cartridges into the system; this means may be a robot arm or the like, controlled by a computer processor executing a suitable computer program. A robot arm may also be used for introducing and/or removing liquid from the system.

According to a yet further aspect of the present invention, there is provided a computer program for automated separation of molecules from a liquid medium, the computer program comprising executable code for controlling the application of current selectively to at least two electrode pairs in a predetermined sequence.

According to a still further aspect of the present invention, there is provided a computer program product, the product comprising executable code recorded on a computer-readable carrier medium, the code comprising executable instructions for controlling the application of current selectively to at least two electrode pairs in a predetermined sequence.

These and other aspects of the present invention will now be described by way of example only and without limitation, with reference to the accompanying drawings, in which:

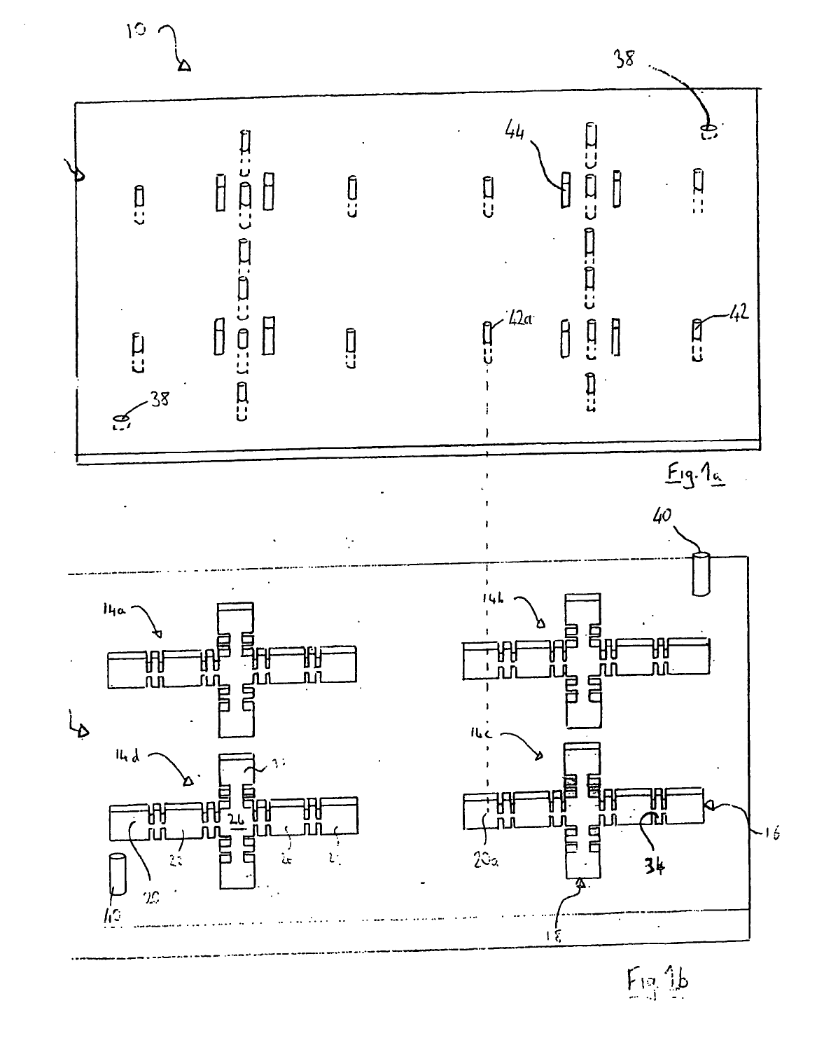

FIGS. 1a and 1b show a cover and a substrate of an apparatus for separation of molecules in solution in accordance with a first embodiment of the present invention;

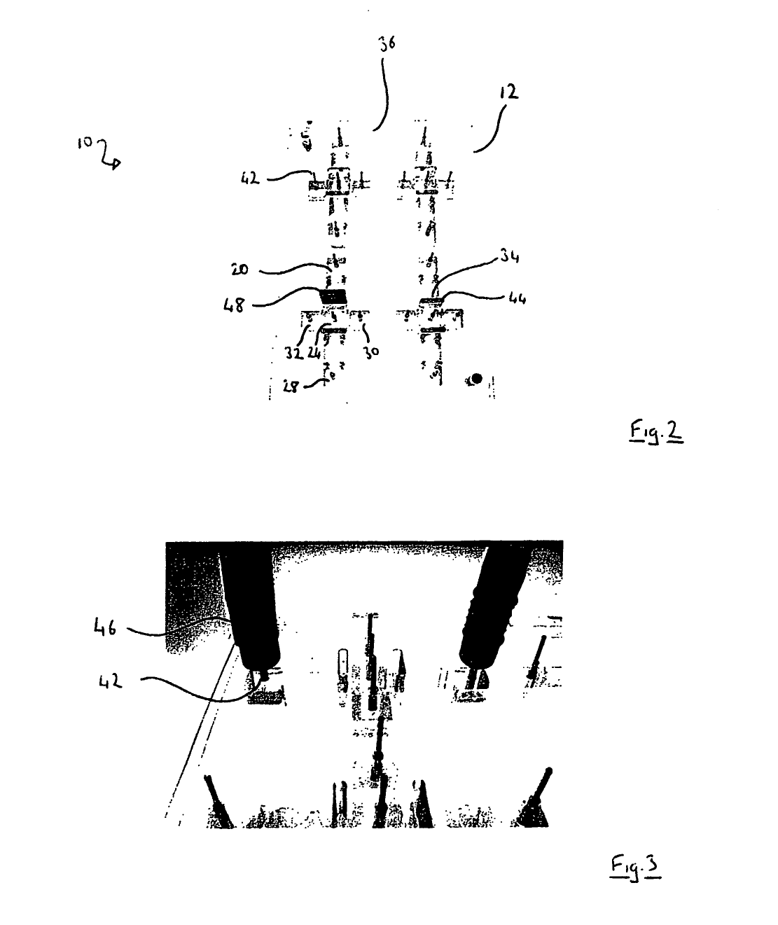

FIG. 2 shows the apparatus of FIG. 1 in an assembled form;

FIG. 3 is a close up view of part of the apparatus of FIG. 1, showing the connection of electrodes thereto;

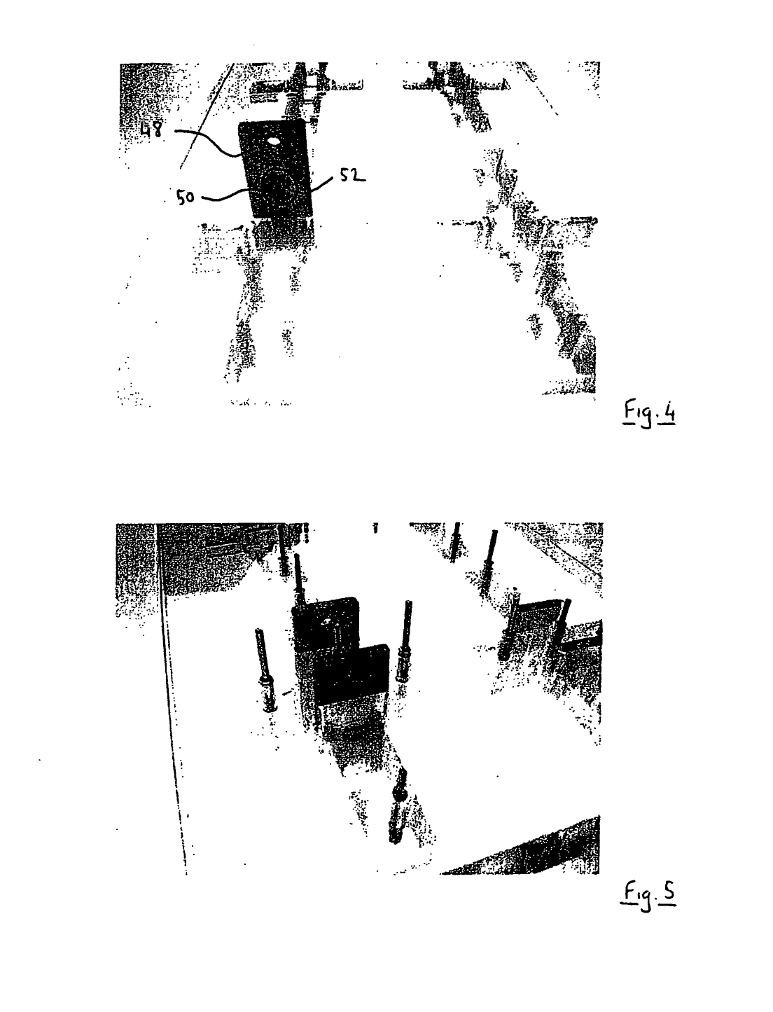

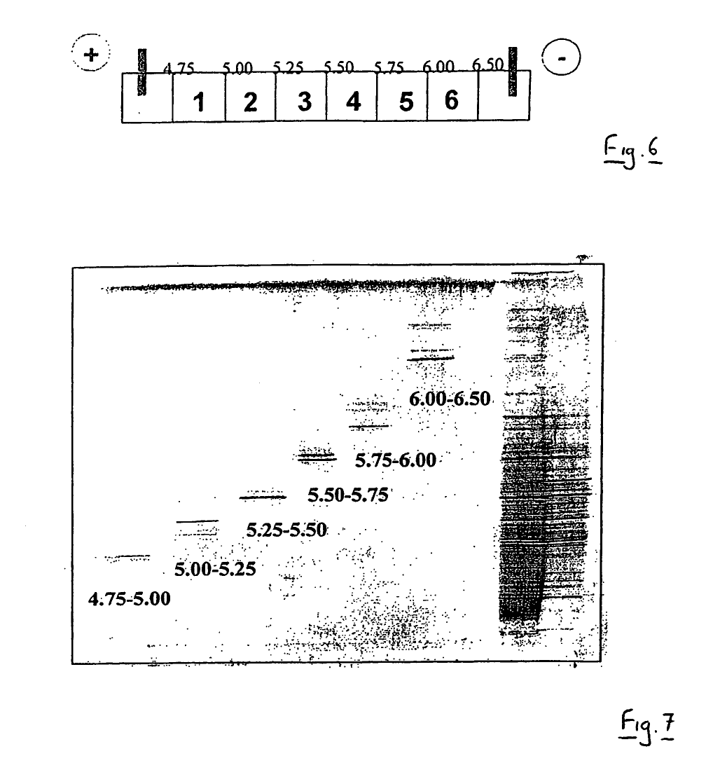

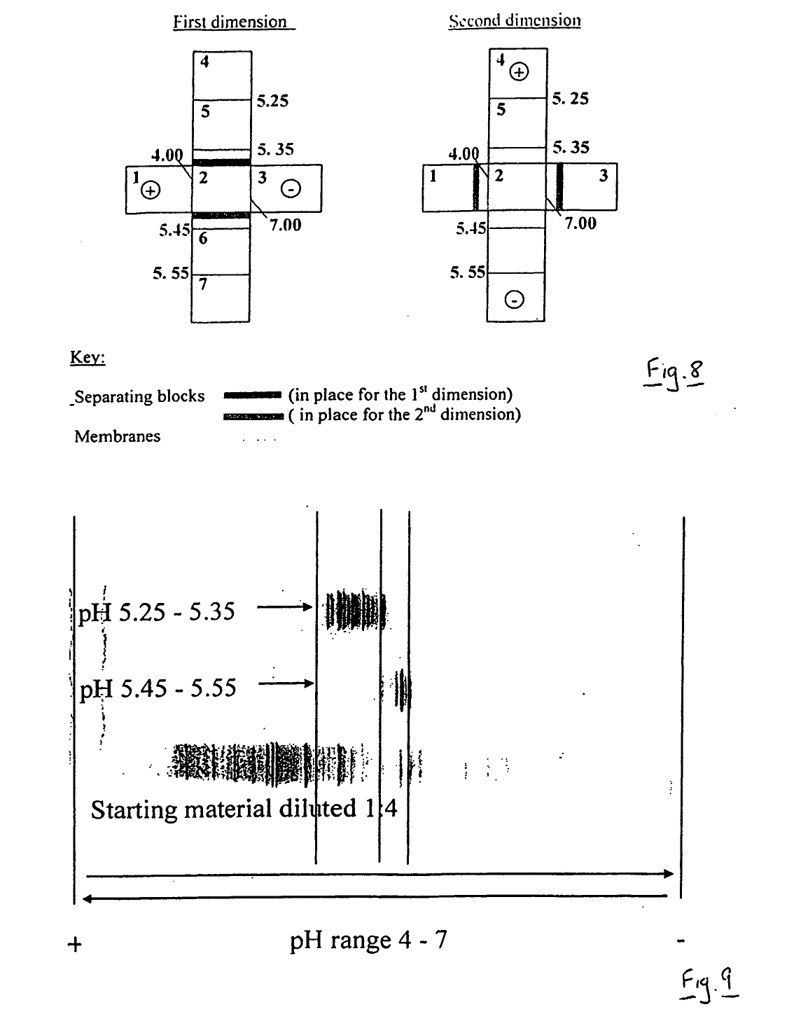

FIGS. 4 and 5 show stages in the preparation for use of the apparatus of FIG. 1;

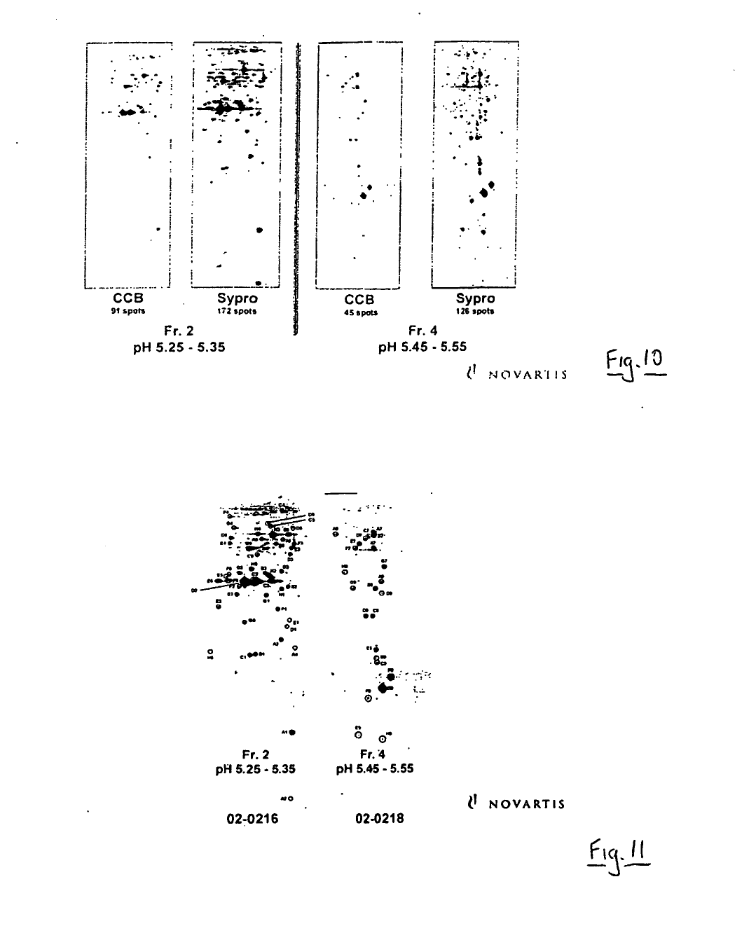

FIG. 6 shows a schematic layout of an apparatus for separation of molecules in solution in a single dimension according to pI;

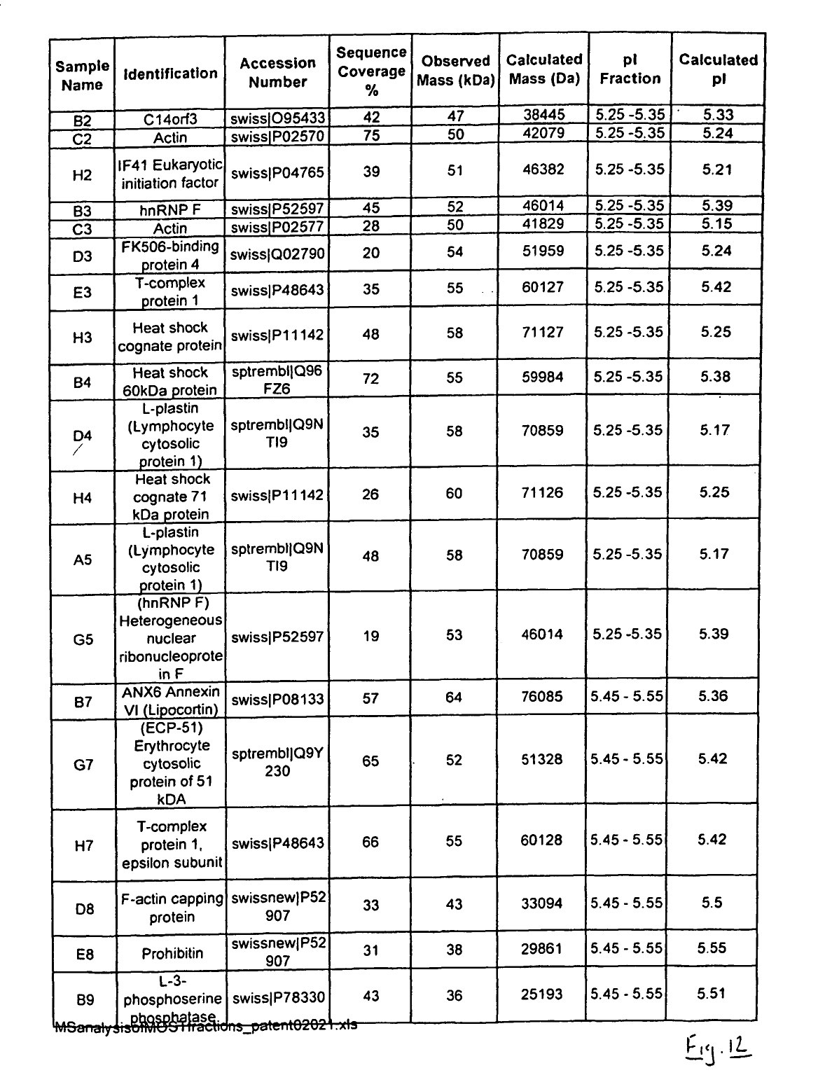

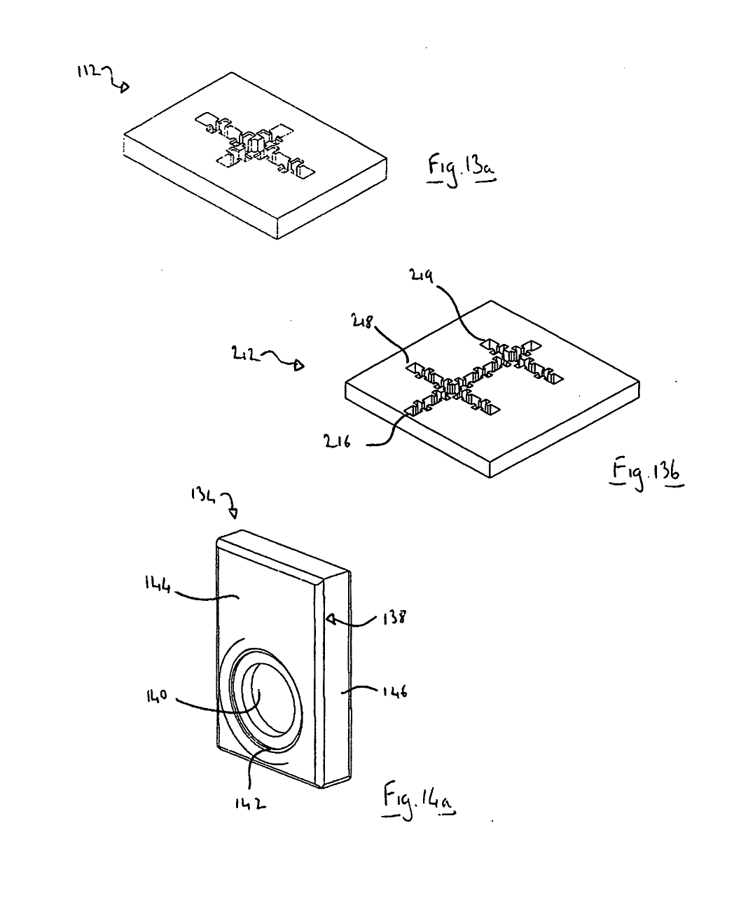

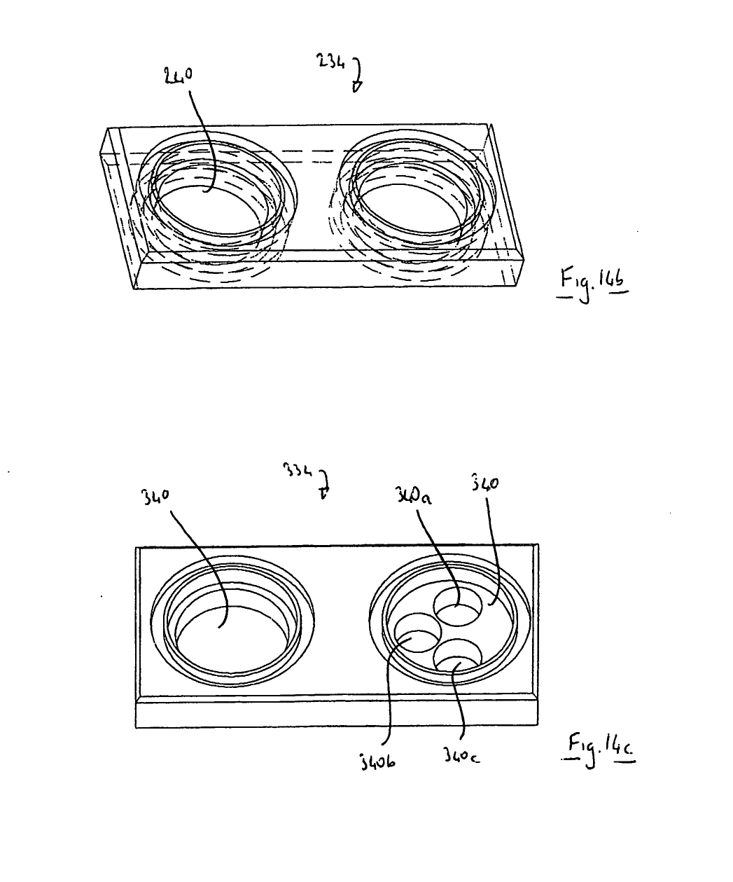

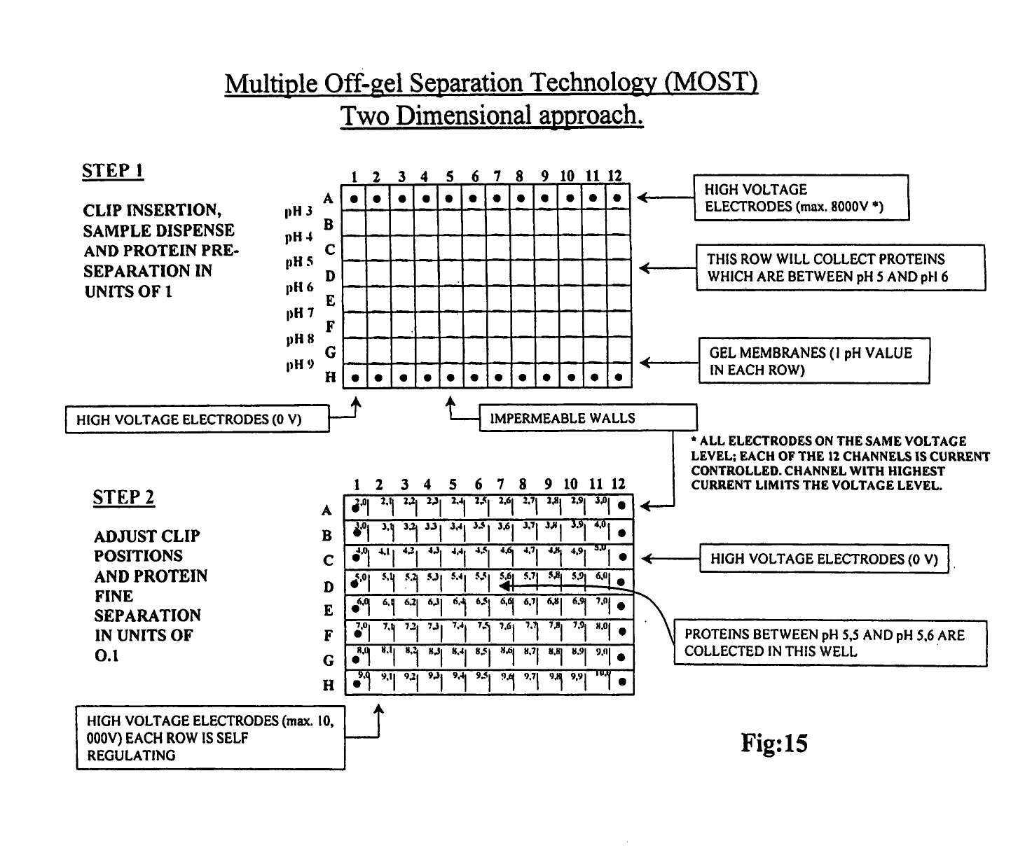

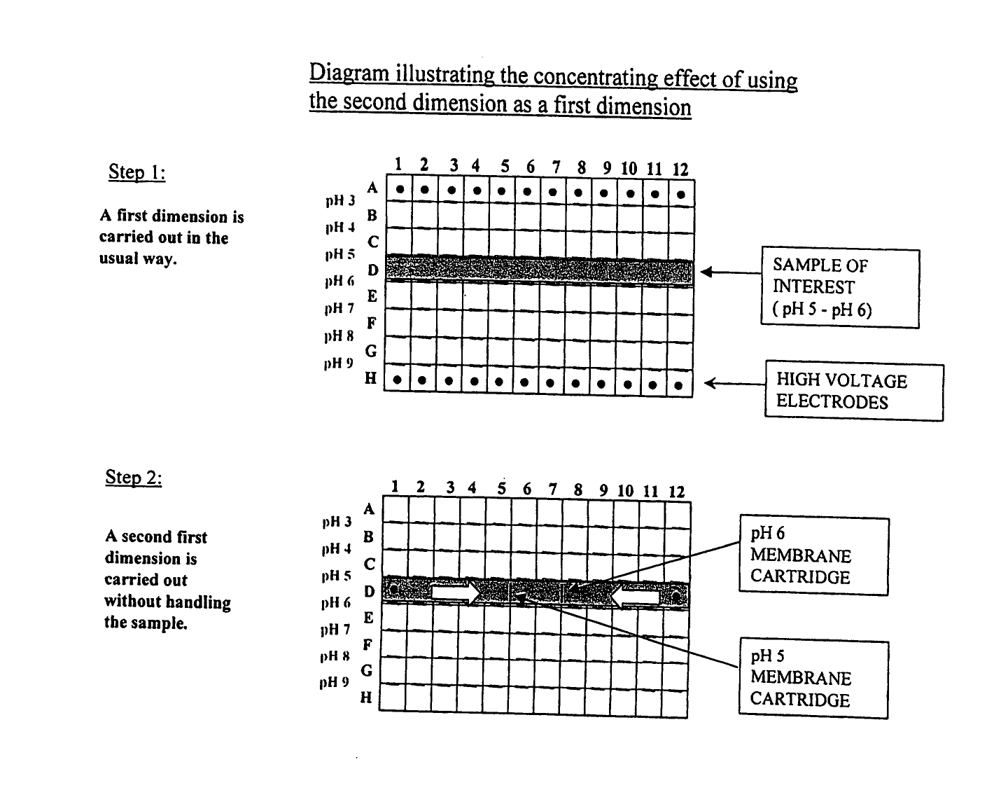

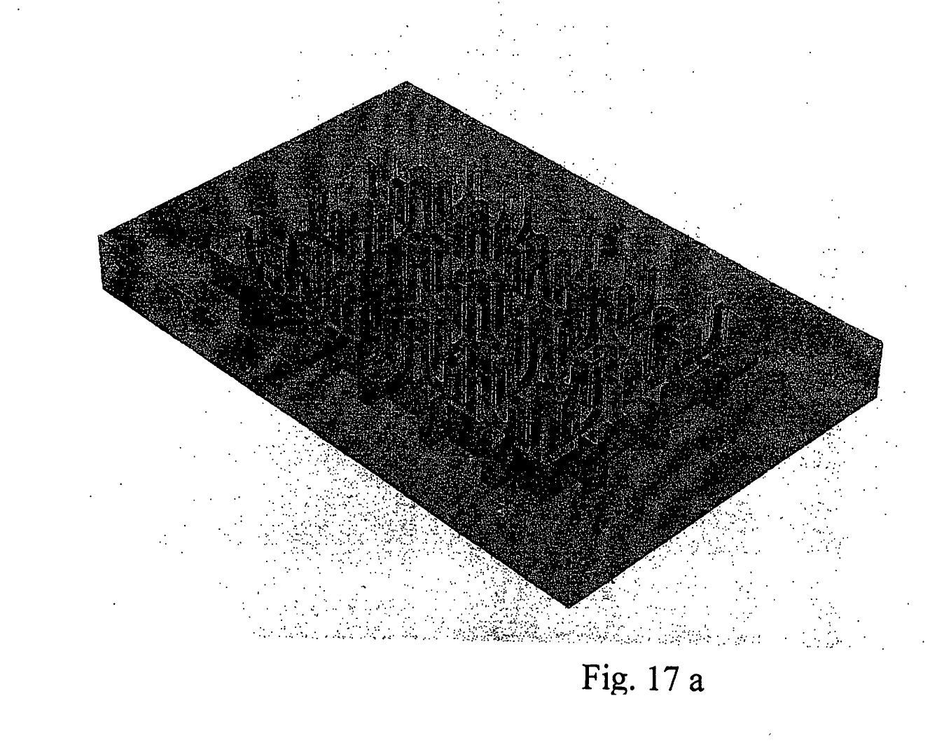

FIG. 7 shows the results of a gel separation of 697 cell line (ATCC DS MZ ACC42) cell extract based on the layout shown in FIG. 6;

FIG. 8 shows a schematic layout of a portion of the apparatus of FIG. 1, prepared for separation of molecules in solution in two dimensions according to pI;

FIG. 9 shows the results of a gel separation of 697 cell line (ATCC DS MZ ACC42) cell extract based on the layout shown in FIG. 8;

FIGS. 10, 11, and 12 show further analysis of the results of the separation shown in FIG. 9. FIGS. 10 and 11 are 2D gel electrophoresis images of 697 cell line (ATCC DS MZ ACC42) cell extract, while FIG. 12 is a table indicating the results of mass spectrometry analysis of selected spots from FIG. 11.

FIGS. 13a and 13b show alternative constructions of apparatus substrates for separation of molecules in solution, in accordance with embodiments of the present invention;

FIGS. 14a, b, c show alternative cartridges for use with the apparatus for separation of molecules in solution of the present invention;

FIG. 15 illustrates a process of multiple dimension separation of molecules in solution using a number of parallel separations;

FIG. 16 shows how the process of FIG. 15 may be used for concentration of samples in solution;

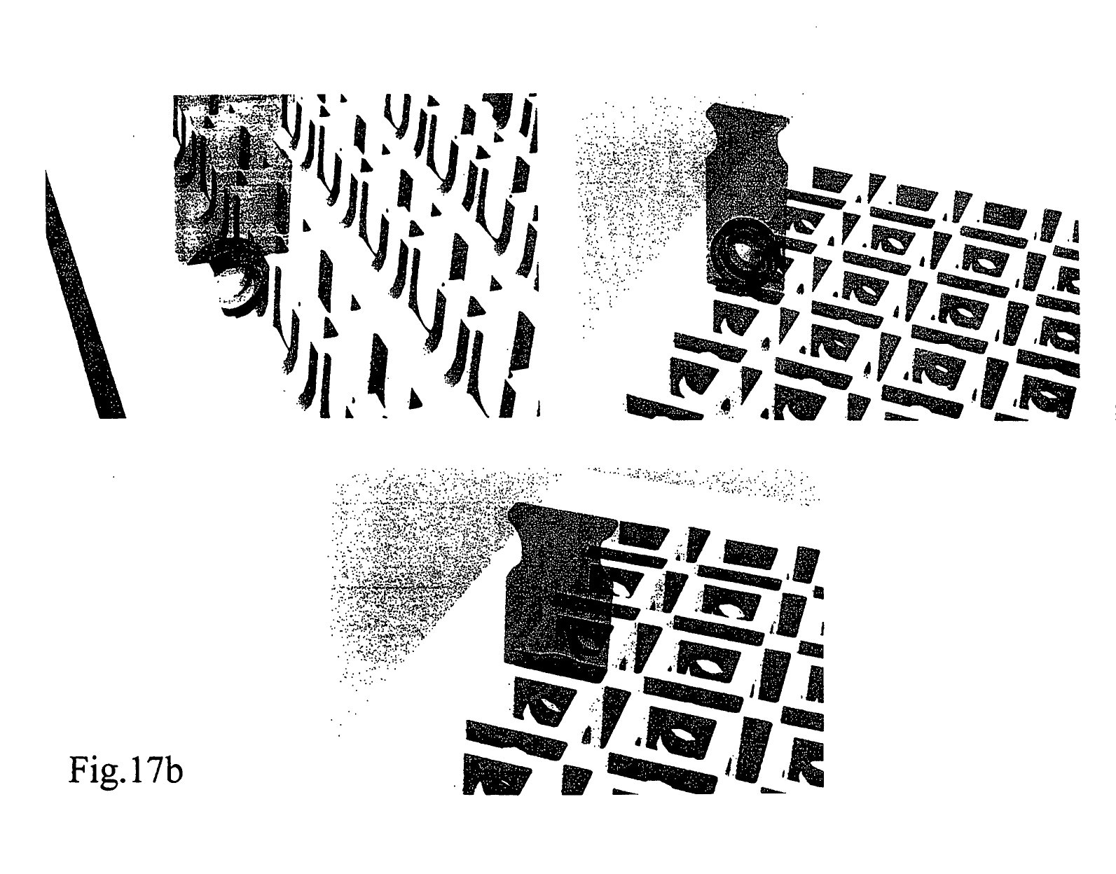

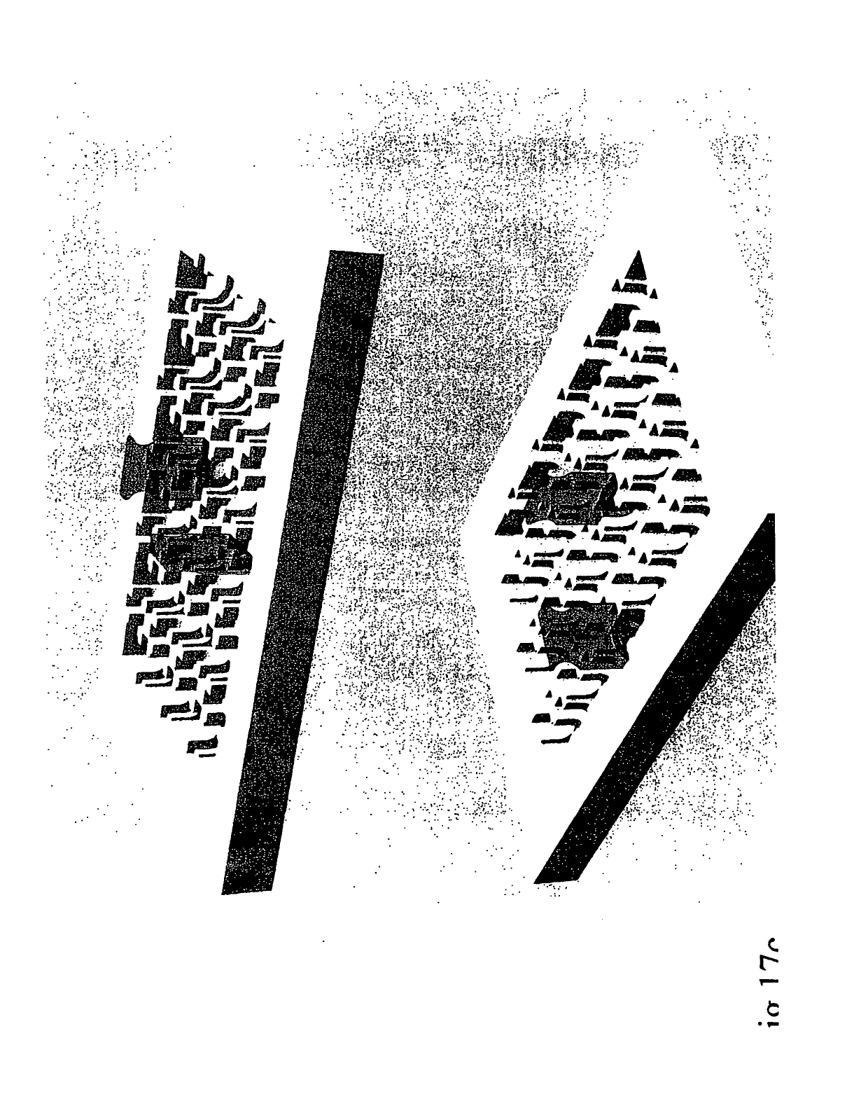

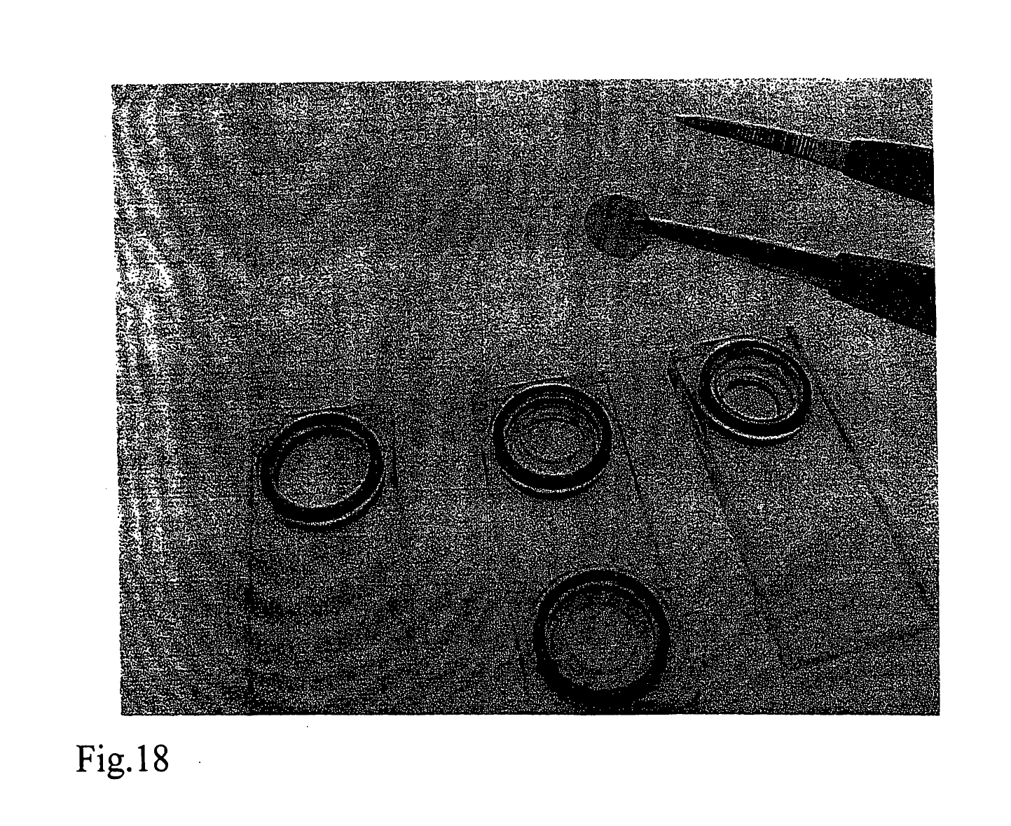

FIGS. 17a, 17b, and 17c illustrate an apparatus which may be used to perform the process of FIGS. 15 and 16;

FIG. 18 is a photograph showing membrane cartridges of the present invention; and

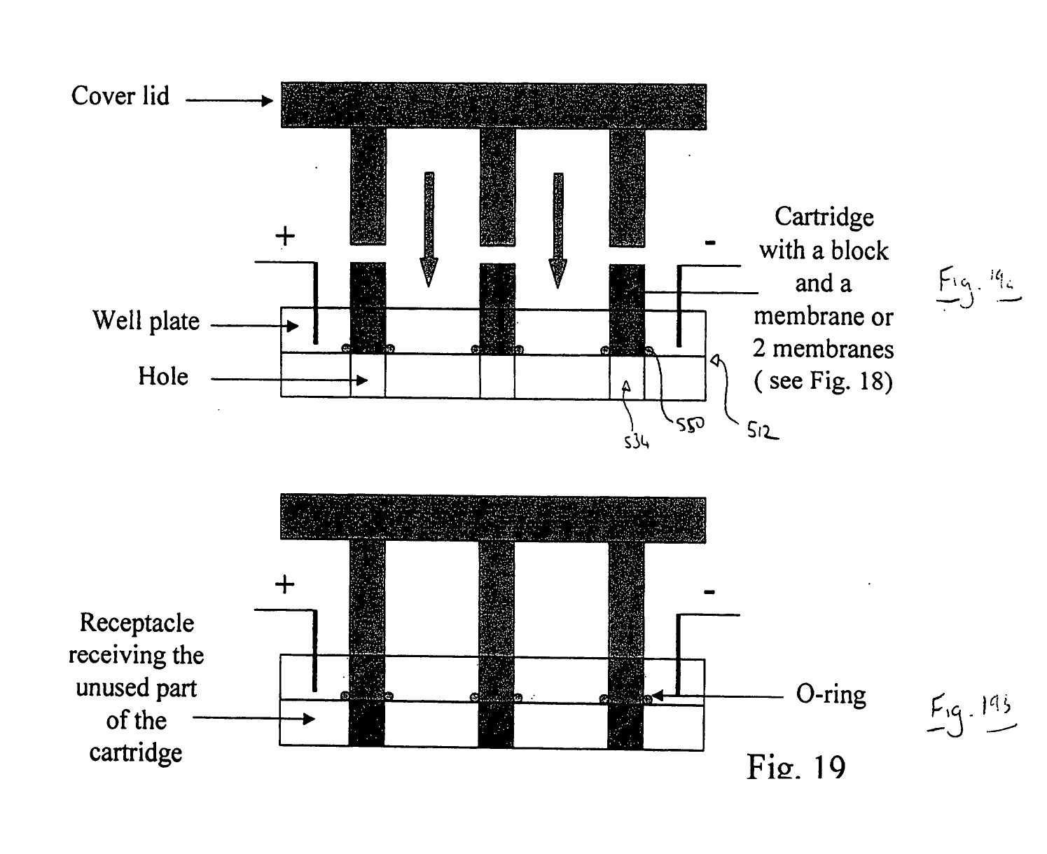

FIG. 19 shows a vertical cross section of a further embodiment of the invention, illustrating how membrane cartridges may be changed during separation.

Referring first of all to FIGS. 1a and 1b, these show a cover 36 (FIG. 1a) and a substrate 12 (FIG. 1b) of an apparatus 10 for separation of molecules in solution in accordance with a first embodiment of the present invention. The apparatus has two parts : a substrate 12 and a cover 36.

The substrate 12 shown has four identical cruciform separation chambers 14a, b, c, d, only one of which will be described in detail, although it will be understood that the description is applicable to all separation chambers. Each separation chamber 14 is formed from a number of compartments or vesicles along first and second perpendicular chamber axes 16, 18. Five separation compartments 20, 22, 24, 26, and 28 are formed along axis 16, and three compartments 30, 24, and 32 along axis 18.

The compartments are interconnected to provide a continuous fluid flow path therethrough. The intersection between each pair of compartments forms a narrow cartridge receptacle 34, which is arranged to receive and retain a cartridge, as will be described later.

The cover 36 is shaped and sized to match the substrate 12, and includes a pair of apertures 38 which cooperate with corresponding protrusions 40 formed on the substrate 12 to locate and hold the cover and substrate in alignment. The cover 36 further includes a number of electrodes 42 arranged such that, when the cover 36 is aligned on the substrate 12, the electrodes 42 extend into certain of the compartments 20, 24, 28, 30, 32 of the substrate 12. In the interests of clarity, electrode 42a extends into compartment 20a, as indicated by the dotted line. When the apparatus is assembled, electrodes 42 are present at either end of the two axes 16, 18, as well as in the central compartment 24.

The cover 36 further includes a number of slots 44 which align with the narrow cartridge receptacles 34 separating the compartments when the cover is fitted on the substrate. These slots allow cartridges to be inserted through the slots into the cartridge receptacles 34 when the cover is in place, as is best illustrated in FIG. 2.

FIG. 2 shows the apparatus 10 in the assembled condition, with the cover 36 located on the substrate 12. The alignment of the electrodes 42 with the compartments 20, 24, 28, 30, 32 can be clearly seen, as can the location of the slots 44 in alignment with the cartridge receptacles 34. FIG. 2 also illustrates the location of a cartridge 48 in one of the slots 44, extending into the cartridge receptacle 34. In use, of course, none, one, or several of the slots 44 and receptacles 34 may have a cartridge 48 located therein. The purpose and precise form of the cartridge 48 will depend on the application to which the apparatus is being put; however, as will be described, in general the cartridge 48 may be selected from either a solid barrier, to prevent communication between adjacent compartments, or a permeable membrane to form a component of the separation procedure. Although in the illustrated embodiment the slots 44 are only present adjacent selected receptacles 34, it will be apparent to the skilled person that the number and location of the slots 44 may be varied to provide slots adjacent fewer or more of the cartridge receptacles 34.

FIG. 3 shows an enlarged view of one of the cruciform chambers 14 of the apparatus of FIG. 2, illustrating the attachment of power supply connections 46 to one of the pairs of electrodes 42.

FIGS. 4 and 5 show further views of the substrate 12 and substrate and cover 36 including a cartridge 48 inserted into the apparatus. The cartridge 48 is dimensioned to fit into the cartridge receptacle 34 of the substrate 12, and includes a circular aperture 50 on the lower portion thereof in which is mounted a polyacrylamide membrane 52, which is permeable to certain molecules. In use, the cartridge 48 is fully inserted into the receptacle 34, such that communication between adjacent compartments is blocked other than through the membrane 52. Further details of the nature and function of the membrane 52 will be given below.

A brief summary of the operation of the apparatus of FIGS. 1 to 7 will now be described, followed by an example of its use. The apparatus is intended for the separation of molecules from solution in accordance with selected properties of those molecules. The apparatus separates molecules over two dimensions, across each of the axes of compartments of the apparatus. Separation in the first dimension is based on preparative isoelectric membrane electrophoresis technology. This is based on the ability of acrylamido buffers (such as Immobiline®, Pharmacia) to become covalently linked to a polyacrylamide gel, fixing the buffering pH of the gel at any desired value. The membranes are placed between the compartments in a graduated pH series (see FIG. 8) separating one compartment from another. Once an electric field is applied to the gradient of pH buffered polyacrylamide membranes, they become “pI selective” and will only allow amphoteric species such as proteins to move towards (not away from) their isoelectric pH. The protein macroions are kept in a reservoir and are continuously exposed to the electric field which is applied across the chemical buffering system. The shape of the chamber is designed in such a manner that an electric field is developed across it, thereby generating a migration flux of the charged species present in the solution. Eventually the sample (or the selected sample mixture) is trapped in a compartment isolated by two membranes (one either side of the compartment) which have pIs encompassing the pI value of the protein in the sample being purified. Therefore, by a continuous titration process all other impurities, either non isoelectric or having different pI values, are forced to leave the compartment. After a period of focusing, compartments containing only the sample or the sample mixture of interest are left. A second dimension of separation is then applied. In the second dimension, the membranes used for isoelectric focusing may be replaced with alternative types of membrane. For example, membranes such as Biodyne® (Pall Corporation), Immunodyne®, Nylon, or nitrocellulose membranes may all be used to bind molecules having particular properties. It will be apparent to the skilled person which membrane should be selected for a particular application. Antibodies or substrates may be affixed to membranes to immobilise desired molecules. In place of membranes, beads such as enzyme immobilisation beads or the like may be used, and incorporated into cavities in cartridges in the apparatus.

To effect the separation in the second dimension, an electric field may be applied across the second axis of compartments. To do this, the electrical connections to the electrodes may be physically reconfigured; alternatively, a switching mechanism may be arranged to switch the electrical supply between electrode pairs. The switching mechanism may be under the control of a suitably programmed computing device. In addition, or instead of, an electric field, the second separation may be aided or effected by agitation of the apparatus, to encourage the molecules in solution to come into contact with the membranes.

An example of use of a prior art apparatus which allows separation of molecules in a single dimension will now be described, in order to provide some background to the operation of the present invention. Subsequently, an example of use of the apparatus of the present invention will be described. The examples illustrate the fractionation of proteins from 697 cell line (ATCC DS MZ ACC42) cell extracts.

Preparation of Isoelectric Membranes

A schematic layout of the prior art apparatus is given in FIG. 6, which shows a single axis of compartments 1 to 6 separated by membranes of differing pH values, as indicated above each membrane. The membranes are produced using a fibreglass Whatman GF/F 4.7cm filter. The pH of these membranes is determined using recipes. We are interested as an example in producing membranes of pH range: 4.75, 5.00, 5.25, 5.50, 5.75, 6.00, 6.50 therefore the recipes used are as follows:

| | Immobiline pK buffers used (μl) | | | | | | | |
|---|---|---|---|---|---|---|---|---|
| | | | | | | | 1 M Tris | 1 M Acetic |
| pH | 3.6 | 4.6 | 6.2 | 7.0 | 8.5 | 9.3 | base (μl) | acid (μl) |
| 4.75 | 332 | 192 | 253 | 49 | 0 | 145 | 55 | 0 |
| 5.00 | 310 | 229 | 235 | 65 | 0 | 190 | 47 | 0 |
| 5.25 | 289 | 267 | 217 | 80 | 0 | 235 | 40 | 0 |
| 5.50 | 268 | 305 | 199 | 95 | 0 | 280 | 32 | 0 |
| 5.75 | 246 | 343 | 181 | 111 | 0 | 325 | 24 | 0 |
| 6.00 | 225 | 381 | 163 | 126 | 0 | 371 | 16 | 0 |
| 6.50 | 182 | 456 | 126 | 157 | 0 | 461 | 0 | 0 |

These figures are based on making 2 membranes of each pH specified.

The membranes are prepared in six separate vesicles. To each of the 6 vesicles add 2 ml of deionised water (ddH20). To these add the acrylamide (Immobiline) buffer volumes as stated in the table. Make all vesicles up to 6 ml in volume using ddH20 and mix. At this point the actual pH of the solutions must be measured, using a pH meter, to ensure a linear gradient has been created; any differences in pH may be rectified by adding either a small amount of acid or basic immobiline, this however should not be necessary and should be avoided if possible. The pH is then adjusted to 6.5±0.2 with 1M Tris base or 1M Acetic acid (in this example 1M Tris base) using the volumes given in the table, and mixed well. Add 3.333 ml of acrylamide/bis stock (30% T) (28.8 g acrylamide+1.2 g bis). Make all vesicles up to 10 ml in volume using ddH20 and mix well.

At this stage the following steps must be taken for each pH solution at a time. Add 5 μl TEMED, 10 μl fresh APS (400 mg/ml), and mix briefly. After these components have been added the membranes should be prepared within 6 minutes, as follows.

Deliver 3 ml of the solution to each of 2 wells in a moulding block. Carefully introduce a Whatman GF/F, 4.7 cm filter paper into each well by lowering it at an angle and allowing it to saturate via capillary action. Once saturated lower the paper into the well, gently pressing it into place with gloved fingers. The filter must settle into the well so that its edges are completely within and evenly centred. Pipette an additional 2 ml of the solution over each of the filter papers. Slowly lower glass covers over each of the two membranes allowing any excess solution to escape. Avoid trapping air bubbles as these may cause holes in the membrane.

Do not disturb the membranes for 20 minutes, then place in an oven at 50° C. for 1 hr or leave at room temperature to polymerise overnight.

After complete polymerisation the membranes can be removed from the wells and placed in 30% ethanol (enough to cover the membranes). These can then be stored at 4° C. for up to one month.

When the membranes are to be used, they are cut into a series of rectangular membranes approximately 8 mm wide and 8 mm long. These are placed in the apparatus, and should fit snugly into each slot to avoid leaking during the focusing step. Both examples given here used membranes placed directly into the slot, rather than by means of a membrane cartridge; however, it is preferred to use cartridges as described, both for convenience, and since during long focusing times there is a small risk of leakage where membranes are used directly in the apparatus.

Setting Up Ready for Focusing

The arrangement is illustrated schematically in FIG. 6. pH membranes are indicated by vertical lines between compartments, and positive and negative electrodes are indicated. All compartments are separated with a membrane. The sample required for focusing is placed in compartments 1 to 6, and all other wells (unnumbered) are filled with Rabilloud buffer.

Sample Preparation

The sample (697 cell line (ATCC DS MZ ACC42) cells 10 mg/ml) is diluted 1:4 with Rabilloud buffer containing 1% DTT. 250 μl of this dilution are pipetted into wells 1 to 6, and the remaining wells (unnumbered), in which are located the positive and negative electrodes, are filled with 250 μl of the Rabilloud buffer.

Focusing

A Multiphor apparatus (Amersham Biosciences) was used for the focusing procedure. The cooling on the Multiphor is set at 8° C. The focusing program is as follows: Conditions 4 hrs, 500V, 2 mA, 2 W; overnight, 100V, 2 mA, 2 W. Total time of 15 hrs.

Analysis of Results

The results were run on a dry IPG gel pH 4-7 and was then stained using the Pharmacia Biotech silver stain kit, (the protocol which contains the TCA fixing step). The starting material (697 cell line (ATCC DS MZ ACC42) cells 10 mg/ml) is diluted 1:4 with Rabilloud buffer to give a good clear reading on the gel. The gel is illustrated in FIG. 7, and shows a clear separation of the 697 cell line (ATCC DS MZ ACC42) cell extract into distinct pH ranges. The right hand lanes were run with whole cell extract.

### EXAMPLE 2

An example of separation of molecules in two dimensions, according to an embodiment of the present invention, will now be described. The membranes are prepared by the method previously mentioned (using Immobiline pK buffers). We used membranes of pH 4.00, 5.25, 5.35, 5.45, 5.55 & 7.00 which were made according to the following table.

| | Immobiline pK buffers used (μl) | | | | | | | |
|---|---|---|---|---|---|---|---|---|
| | | | | | | | 1 M Tris | 1 M Acetic |
| pH | 3.6 | 4.6 | 6.2 | 7.0 | 8.5 | 9.3 | base (μl) | acid (μl) |
| 4.00 | 276 | 103 | 59 | 0 | 0 | 170 | 42 | 0 |
| 5.25 | 300 | 225 | 124 | 0 | 0 | 372 | 24 | 0 |
| 5.35 | 302 | 235 | 130 | 0 | 0 | 388 | 22 | 0 |
| 5.45 | 304 | 244 | 135 | 0 | 0 | 404 | 21 | 0 |
| 5.55 | 306 | 254 | 140 | 0 | 0 | 420 | 19 | 0 |
| 7.00 | 139 | 532 | 90 | 188 | 0 | 551 | 0 | 9 |

These figures are based on making 2 membranes of each pH specified.

A schematic layout of the apparatus is illustrated in FIG. 8. The apparatus contains seven compartments, numbered 1 to 7, arranged in two intersecting axes. Compartments 1 to 3 form the first axis, horizontal in the Figure, while compartments 4, 5, 2, 6, and 7 form the second, vertical axis. Each separation step takes place with a different configuration of the apparatus; the first step, shown in the left-hand part of FIG. 8, takes place along the first axis with separating blocks placed to isolate the second axis, while the second step, shown in the right-hand part of FIG. 8, takes place along the second axis with separating blocks placed to isolate the first axis.

All seven compartments are equilibrated in Rabilloud buffer containing DTT for one hour. After this time the first dimension can proceed.

First Dimension

The separating blocks are in place as shown in the left-hand part of FIG. 8, the positive electrode is placed in well 1, negative in well 3. Finally, 300 μl of starting material can be placed in well 2, all other wells remain filled with the Rabilloud buffer. The program for the first dimension is as follows: 200V, 2 mA, 2 W, 10 minutes; 500V, 2 mA, 2 W, 2 hrs. After focusing the samples in wells 1,2 & 3 are removed and stocked. The second dimension then proceeds.

Second Dimension

The separating blocks are removed from the positions shown in the first diagram and are replaced as shown in the right-hand part of FIG. 8. Wells 1 and 3 are refilled with 300 μl of Rabilloud buffer, well number 2 can then be refilled with the separated material from the first separation, and the Rabilloud buffer in wells 5 and 6 can be removed and replaced with separated material. The positive electrode is placed in well number 4 and the negative in well number 7. The program for the second dimension is as follows: 500V, 2 mA, 2 W, 2 hrs minimum; 1000V, 2 mA, 2 W, 2 hrs. The samples can then be run on a Dry IPG gel pH 4-7.

Visualising the Results

Rehydration

a) Mark the cathodic side of the gel
b) Wet a clean thick glass plate with a few drops of water and place the gel on the plate, gel side up
c) Using a clean rubber roller, roll the gel to remove any trapped air bubbles
d) Mount the gel in the cassette taking special care that the U-frame gasket also seals over the cut-off corner of the supporting plastic foil and that the clamps are mounted correctly to avoid leakage
e) Fill the cassette with the desired amount of Rabilloud buffer and leave to rehydrate for 2 hrs.
   Equilibration
a) After this time remove the gel and place it on the Multiphor ready for focusing ensuring that there are no air bubbles trapped between the gel and the cooling plate.
b) Remove any excess liquid by gently pressing it with filter paper.
c) Place two filter strips dampened with Rabilloud buffer or ddH2O along the length of the gel on opposite sides, again removing any excess buffer by gently pressing on them with filter paper. Cut off any parts of the strip which protrude beyond the short ends of the gel using sharp scissors. These strips ensure good electrical contact between the gel and the electrodes. They also prevent sparking and allow salt ions from the gel to migrate into the strips.
d) Equilibrate the gel at 500V, 5 mA, 5 W for a minimum of 45 minutes.

Sample Loading

Using sample application pieces, allow them to absorb 20 μl of sample via capillary action. Place the pieces along the right hand length of the gel proportionally spaced from one another. The focusing program is as follows.

| Voltage 1 | Voltage 2 | Time (hr) | Amps (mA) | Watts (W) |
|---|---|---|---|---|
| 0 | 500 | 20 | 5 | 5 |
| 500 | 1000 | 2 | 5 | 5 |
| 1000 | 2000 | 3 | 5 | 5 |
| 2000 | 3500 | 0.5 | 5 | 5 |

After focusing the gel is stained using Pharmacia Biotech silver stain kit. The starting material (697 cell line (ATCC DS MZ ACC42) cells 10 mg/ml) was diluted 1:4 to show a good resolution upon the IPG gel. As can be seen from the results in FIG. 9, there is a clear separation between the two samples of differing pH range, showing a fine and distinct separation range, superior to that obtained with a one dimensional separation as shown in FIG. 7.

A further analysis of the results of the separation was conducted using two dimensional electrophoresis. Samples of the 697 cell line (ATCC DS MZ ACC42) protein separation were taken from wells 2 and 4 (see FIG. 8); 125 μl from fraction 2 and 140 μl from fraction 4. The two samples were divided into two and diluted with buffer so that each sample rehydrated two IPG strips pH 5-6. These four IPG strips were put under the same standard first dimension focusing conditions (see table) and were then loaded onto four separate 12% SDS 2-D gels. Once again all four gels underwent identical programs for the second dimension.

| | | | Volthour |
|---|---|---|---|
| V start | V end | Time (h) | product (Vh) |
| 0 | 300 | 0.01 | 1.5 |
| 300 | 300 | 3 | 900 |
| 300 | 3500 | 5 | 9500 |
| 3500 | 3500 | 20 | 70000 |
| | Total | 28.01 | 80401.5 |

Out of the two gels per sample one was stained using BioRad Sypro Ruby (‘Spyro’ on FIG. 10) and the other with BioRad Colloidal Coomassie Blue stain (‘CCB’ on FIG. 10). FIG. 10 shows the gels, with each spot indicating an isolated protein from the cell extract.

The two CCB stains are shown again in FIG. 11, with a number of isolated spots marked on each gel. The spots outlined in FIG. 11 from the two Coomassie blue stained gels (one from fraction 2: pH 5.25-5.35 and one from fraction 4 : pH 5.45-5.55) were picked and digested. The samples were then taken to be analysed via mass spectrometry. The results of this are given in FIG. 12. The list of proteins given in the Figure correspond to selected proteins identified by mass spectrometry which are contained in the two fractions. The results indicate that the calculated pI for each protein matches the specific pH boundaries of the sample fraction from which it is derived.

It can be seen that the present invention provides a method and an apparatus whereby samples in solution may be separated over two or more dimensions without the need for intermediate processing or recovery steps. This allows for more rapid separation of molecules, with less user involvement.

FIGS. 13a and 13b illustrate alternative forms of substrate 112, 212 which may be used with the present invention. The substrate 112 shown in FIG. 13a is similar to that of FIG. 1b, although only a single cruciform arrangement is present. The substrate 212 shown in FIG. 13b differs in that three axes 216, 218, 219 of compartments are provided. Two of the axes 218, 219 are parallel to one another, and are perpendicular to the third axis 216. It will also be noted that although axis 218 is symmetrically disposed about axis 216, with two compartments on either side of the axis 216, axis 219 is asymmetric, having a single compartment to one side of the axis 216, and two compartments to the other side of the axis.

FIGS. 14a, 14b, and 14c show alternative forms of cartridge 134, 234, 334 which may be used with the apparatus of FIG. 1. Referring only to FIG. 14a for now, the cartridge comprises a plastics frame 138 shaped and dimensioned so as to cooperate with the slots and cartridge receptacles provided on the substrate and cover of the apparatus. The frame 138 defines a circular aperture 140 which is grooved 142 to receive an elastomeric O-ring, which in use retains a disc of permeable membrane (not shown) across the aperture 140. The upper portion 144 of the cartridge 134 is left blank, and may be used with the cartridge in an inverted orientation to isolate adjacent compartments from one another. The upper portion 144 or the side faces 146 of the cartridge 134 may be used for handling the cartridge with forceps or by hand.

The other two cartridge forms (FIGS. 14b and 14c) 234, 334, are similar in construction, although a second aperture 240, 340 is provided in place of the blank upper portion 144 of the cartridge of FIG. 14a. This may be used to retain a second permeable membrane, of the same or different type from the first membrane, to allow the membrane to be rapidly changed when the apparatus is in use, by simply inverting the cartridge. A further modification is made to the third cartridge 334, in that one of the apertures 340 contains three separate apertures 340a, 340b, 340c. In use, each of these apertures 340a, 340b, 340c may contain a different membrane, thereby allowing distinct types of membrane to be presented to the same solution during a separation.

FIG. 15 illustrates a process according to the present invention which may be used to provide multiple parallel separation of multiple samples, in two dimensions. The apparatus 410 is in the form of a rectangular array of compartments, each of which is connected to its neighbours. Impermeable barriers or permeable membrane cartridges may be located between adjacent compartments, as described above. During the separation, the first dimension of separation may be used to separate samples across a relatively coarse gradient (in this example, steps of 1 pH unit in the vertical direction). Once this has been done, the second dimension is applied in the perpendicular direction across a finer gradient; in this example, steps of 0.1 pH unit in the horizontal direction. The final result provides a finely separated series of fractions, with a different fraction in each well of the apparatus.

FIG. 16 illustrates a further variation of this method. In this example, the first dimension of separation is carried out in the usual way, with multiple samples being separated across a gradient with steps of 1 pH unit in the vertical direction. The second dimension, however, is carried out using all the samples from a particular pH range, which are then separated across the same particular pH range within a smaller volume. This has the effect of concentrating the desired fraction into one or a few compartments, without further user intervention being necessary.

FIG. 17a, 17b, and 17c illustrate perspective and close-up views of a twenty-four compartment apparatus suitable for use with the methods described in connection with FIGS. 15 and 16. It can be seen that each compartment is adjacent a cartridge receiving aperture on each side (apart from those compartments at the edges of the apparatus), which leads to a further compartment. FIG. 17b shows two variations of the apparatus, each having slightly different forms of compartment. FIG. 17c illustrates cartridges inserted into the apparatus in both orientations.

FIG. 18 is a photograph of three different types of membrane cartridge, similar to those shown in FIGS. 14a and 14b.

FIG. 19 is a vertical cross section view of a further variation of the invention. In this embodiment, the base of the apparatus 512 includes cartridge-receiving apertures 534 extending completely through the base 512. These apertures allow cartridges to be inserted into the base 512 as shown in FIG. 19a, that is not extending completely into the apertures 534, to present a first membrane to the solution; and thereafter pushed through into the lower portion of the apertures 534 as shown in FIG. 19b, to present the upper portion of the cartridge (whether this is a further membrane or a solid barrier portion) to the solution. To prevent leakage of solution from the compartments, an O-ring 550 is provided around each aperture 534.

1. A method for separating molecules in a liquid medium, the method comprising the steps of :
locating a liquid medium containing molecules to be separated in a series of first fluid compartments, the first compartments being separated along a first axis by pH membranes to form a pH gradient along the series of fluid compartments, and at least one of said first compartments being adjacent a second compartment, said first and second compartment forming a second axis substantially perpendicular to the first axis;
applying a first electric field to the liquid medium along a first axis of the fluid compartments, thereby causing charged molecules in the liquid medium to migrate to their isoelectric point along the first axis; and
applying a second electric field to the liquid medium along said second axis, thereby causing charged molecules in the liquid medium to separate along said second axis according to a second characteristic of the molecules.

2. The method of claim 1, wherein the second electric field is applied sequentially to the first electric field.

3. The method of claim 1, wherein the compartments along the second axis are separated by membranes.

4. The method of the preceding claim, wherein the second axis is formed by a plurality of second compartments adjacent the first compartment.

5. The method of the preceding claim, wherein a plurality of first compartments are located adjacent second compartments.

6. The method of the preceding claim further comprising the step of removing impermeable barriers separating the compartments of the second axis from those of the first axis prior to applying the second electric field.

7. The method of claim 6, further comprising the step of replacing the impermeable barriers with membranes.

8. The method of the preceding claim further comprising the step of separating the first compartments along the first axis from the second compartments of the second axis by means of impermeable barriers prior to applying the second electric field.

9. The method of the preceding claim further comprising the step of applying an electric field to the liquid medium across a third axis substantially perpendicular to the second axis.

10. The method of the preceding claim further comprising the step of recovering one or more separated fractions from one or more compartments.

11. An apparatus for separating molecules in a liquid medium, the apparatus comprising a substrate defining a plurality of first fluid compartments arranged along a first axis, the first compartments being separated by pH membranes to form a pH gradient; at least one of the first compartments having a second compartment disposed adjacent thereto, said first and second compartments defining a second axis substantially perpendicular to the first axis; and at least two electrode pairs disposed across the first and second axes.

12. The apparatus of claim 11, wherein the compartments of the second axis are also separated by membranes.

13. The apparatus of claim 11, wherein the second compartments of the second axis are separated from the first compartments of the first axis by removable impermeable barriers.

14. The apparatus of claim 11, wherein the second axis comprises a plurality of second compartments disposed adjacent the or each of the first compartments of the first axis.

15. The apparatus of claim 11, wherein a plurality of first compartments of the first axis have second compartments disposed adjacent thereto, to provide a plurality of second axes.

16. The apparatus of claims 11, further comprising additional compartments disposed parallel to the first axis, to provide additional separation axes.

17. The apparatus of claim 11, further comprising a cover for location on the substrate.

18. The apparatus of claim 17, wherein the cover comprises means for engaging with the electrode pairs to provide electrical connections thereto.

19. The apparatus of claim 17, wherein the electrode pairs are provided on the cover, such that they extend into respective compartments provided on the substrate.

20. The apparatus of claim 11, further comprising fluid inlet and outlet channels.

21. The apparatus of claim 11, further comprising means for supplying electric current to the electrode pairs, to apply an electric field to the apparatus.

22. The apparatus of claim 21, wherein the current-supplying means is selectively controllable.

23. An apparatus for separation of molecules in a liquid medium, the apparatus comprising a substrate defining a plurality of first fluid compartments arranged along a first axis, at least one of the first compartments having a second compartment disposed adjacent thereto, said first and second compartment defining a second axis substantially perpendicular to the first axis; the first compartments along the first axis having means therebetween for receiving pH membranes to separate the first compartments; and the apparatus further comprising means for receiving at least two electrode pairs disposed along the first and second axes respectively.

24. The apparatus of claim 23, further comprising means for receiving membranes located between the compartments of the second axis.

25. The apparatus of claim 24, wherein the means for receiving membranes comprises means for receiving a removable cartridge holding a membrane.

26. The apparatus of claim 23, further comprising one or more removable cartridges holding a membrane, located within the means for receiving membranes.

27. The apparatus of claim 23, further comprising one or more fluid impermeable barriers of suitable dimensions to be received within the means for receiving membranes.

28. The apparatus of claim 23, further comprising a cover for location on the substrate.

29. A cartridge for use with an apparatus for separating molecules in a liquid medium, the cartridge comprising a frame defining an aperture, said aperture having a membrane therein, the frame further comprising a handling portion to permit handling of the cartridge.

30. The cartridge of claim 29, wherein the frame comprises two co-operating sections, which in use hold the membrane therebetween.

31. The cartridge of claim 29, wherein the frame further defines a second aperture having a second membrane therein.

32. The cartridge of claim 29, further comprising polymeric or elastomeric seals for sealing the edges of the membrane from fluid penetration.

33. An automated system for separation of molecules from a liquid medium, the system comprising:
a substrate defining a plurality of first fluid compartments arranged along a first axis, the first compartments being separated by pH membranes to form a pH gradient; at least one of the first compartments having a second compartment disposed adjacent thereto, the first and second compartments defining a second axis substantially perpendicular to the first axis; and at least two electrode pairs disposed across the first and second axes;
control means for applying current selectively to the electrode pairs in a predetermined sequence; and
means for introducing and removing liquid containing molecules to be separated to and from the chambers.

34. A computer program for automated separation of molecules from a liquid medium, the computer program comprising executable code for controlling the application of current selectively to at least two electrode pairs in a predetermined sequence.
